(19) United States
(12) Patent Application Publication
(10) Pub. No.: US 2002/0106408 A1
(43) Pub. Date: Aug. 8, 2002

(54) Prolamin-based sustained-release compositions and delayed-onset compositions

(76) Inventors: Johnatan Bacon, Montreal (CA); Alain Desjardins, Montreal (CA)

Correspondence Address: PENNIE AND EDMONDS, 1155 AVENUE OF THE AMERICAS, NEW YORK, NY, 100362711

(21) Appl. No.: 09/728,840

(22) Filed: Dec. 1, 2000

(51) Int. Cl. A61K 9/24; A61K 9/16; A61K 9/50

(52) U.S. Cl. 424/471; 514/469; 514/629; 514/179; 514/355; 514/202; 514/165

(57) ABSTRACT

In one embodiment, the invention relates to sustained-release compositions comprising one or more prolamins, one or more gelling agents, and one or more active agents. Such compositions are particularly useful for controlled delivery of high solubility and/or high dosage active agents. In another embodiment, the present invention relates to delayed-onset compositions comprising a dry coating comprising one or more prolamins and one or more gelling agents.

### FIELD OF THE INVENTION

This invention relates to novel solid sustained-release compositions comprising a prolamin, a gelling agent, and an active agent in the core of the sustained-release compositions. This invention also relates to delayed-onset compositions comprising a dry compressed coating which is comprised of a prolamin and a gelling agent.

### BACKGROUND OF THE INVENTION

One of the critical factors influencing the rate of absorption of an active agent administered as a tablet or other solid dosage form is the rate of dissolution of the dosage form in the body fluids of a human or animal.

This factor is the basis for the so-called controlled-release, extended-release, sustained-release or prolonged-action pharmaceutical preparations that are designed to produce slow, uniform release and absorption of the active agent over a period of hours, days, weeks, months or years. Advantages of controlled-release formulations are a reduction in frequency of administration of the drug as compared with conventional dosage forms (often resulting in improved patient compliance), maintenance of a therapeutic effect over a set period of time, and decreased incidence and/or intensity of undesired side effects of the active agent by elimination of the peaks in plasma concentration that often occur after administration of immediate-release dosage forms.

Many systems have been proposed and developed as matrices for the release of active agents. For example, polymeric materials such as polyvinyl chloride, polyethylene amides, ethyl cellulose, silicone and poly (hydroxymethyl methacrylate), have been proposed as vehicles for the slow release of drugs. See U.S. Pat. No. 3,087,860 to Endicott et al.; U.S. Pat. No. 2,987,445 to Levesque et al.; Salomon et al., Pharm. Acta Helv., 55, 174-182 (1980); Korsmeyer, Diffusion Controlled Systems: Hydrogels, Chap. 2, pp 15-37 in Polymers for Controlled Drug Delivery, Ed. Tarcha, CRC Press, Boca Raton, Fla. USA (1991); Buri et al., Pharm. Acta Helv. 55, 189-197 (1980).

Despite the great variety of known sustained release compositions, scientists are constantly searching for novel sustained-release compositions to broaden the spectrum of sustained-delivery options, and to deliver a wide variety of active agents in a consistent and reliable manner. As more and more active/pharmaceutical agents become available, new challenges are often presented and new solutions are needed.

One of such challenges presented relates to pharmaceutical agents that need to be delivered in high dosages and/or are highly soluble in aqueous solutions. When the dosage is high or when the solubility is high, the amount of excipient material that is required to achieve sustained-release is also high. As a result, the size of the dosage unit often becomes excessive that it becomes problematic to administer the pharmaceutical agent.

Prolamins, particularly zein, previously have been used in controlled-release drug compositions. However, as detailed below, the potential of prolamins as components for sustained-release compositions or delayed-onset compositions has not been fully explored.

Prolamin has been used as a coating component in dosage units. For example, U.S. Pat. No. 4,137,300 to Sheth et al. relates to a sustained-action dosage form of the type comprising a core-mixture of a pharmacologically effective substance and at least two members selected from a higher alkanol and alkanoic acid melting above 25° C. and an outer layer of a prolamin, wherein the ratio of alkanol: alkanoic acid: prolamin vary from about 100:5:3 to about 100:200:80. U.S. Pat. No. 4,876,094 to Benton et al. discloses a dual coating to be applied over controlled-release cores to form dosage forms comprising fats melting at less than approximately 101° F. overcoated with cellulose acetate phthalate or zein. U.S. Pat. No. 5,004,614 to Staniforth discloses a device for controlled-release of an active agent, comprising a core comprising an active agent and a release modifying agent; and an outer coating containing zein covering said core. U.S. Pat. No. 5,160,742 to Mazer et al. discloses sustained-release particles comprising an active ingredient disposed in a core which has at least one coating of a prolamin and one coating of enteric compound thereon. U.S. Pat. Nos. 5,266,331, 5,549,912, and 5,656,295 to Oshlack et al. disclose coating an oxycodone composition with a film composition comprising zein. U.S. Pat. No. 5,356,467 to Oshlack et al discloses using stable aqueous dispersions of zein as controlled-release coating of pharmaceutical, animal, health or food production in an inorganic solvent-free environment. U.S. Pat. Nos. 5,411,745 and 6,077,533 to Oshlack et al discloses zein-coated powder-layered oral dosage forms of morphine. U.S. Pat. No. 5,418,010 to Janda et al. relates to microencapsulating core material in a water-insoluble protein coating such as caseinate, soy concentrate, soy isolate, soy flour, wheat gluten, egg albumen, milk albumen, gelatin, zein, rice gluten, wheat gluten, barley gluten, oat gluten, rye gluten, and sorghum gluten. U.S. Pat. No. 5,500,227 to Oshlack et al. discloses a sustained-release tablet for oral administration comprising an immediate-release tablet core including an insoluble therapeutically active agent having an aqueous solubility of less than or equal to about 5 mg/ml and a sustained-release film coating comprising a hydrophobic material such as zein. U.S. Pat. Nos. 5,603,967 and 5,846,566 to Burguiere et al. disclose controlled-release microcapsules of acetylsalicylic acid which comprise particles of acetylsalicylic acid coated with a coating material comprising zein as a film-forming polymer. U.S. Pat. No. 5,840,332 to Lemer discloses a drug delivery formulation for localized drug release in the gastrointestinal tract of an animal comprising a core comprising a drug and core material and a coating surrounding said core, said coating having an outer surface, wherein the coating comprises water-insoluble hydrophilic particulate matter embedded in a water-insoluble carrier such as zein. U.S. Pat. No. 5,882,715 to Nielsen et al. relates to adding a moisture resistant layer containing zein between the material to be coated and an enteric coating. U.S. Pat. No. 5,958,459 to Chasin et al discloses sustained-release opioid formulations containing a coating which includes zein. U.S. Pat. No. 5,968, 551 to Oshlack et al. discloses a sustained-release analgesic dosage form consisting essentially of inert pharmaceutically acceptable beads coated with an analgesically effective amount of an opioid analgesic or a salt thereof, wherein the beads further comprising a sustained-release overcoat comprising an effective amount of a hydrophobic material such as zein. U.S. Pat. No. 6,024,982 to Oshlack et al. discloses using zein as a coating material for a tablet core containing an insoluble active agent. WO099/20745 to Kim et al. discloses an enteric coated granule first coated with a water-miscible coating material and with a second coating with the a controlled-release coating material. It discloses that the water-miscible first coating material could be sodium alginate, alginic acid, polymethylmethacrylate, wheat protein, soy protein, methylcellulose, hydroxypropylcellulose, hydroxypropylmethylcellulose, polyvinylacetatephthalate, gums (guar gum, locust bean gum, xanthan gum, gellan gum, etc.) and that the controlled-release second coating material could be corn protein extract or prolamin, sodium alginate, alginic acid, guar gum, locust bean gum, zanthan gum, gellan gum, arabic gum etc. It is important to note that none of the above-mentioned references teaches a dry compressed coating containing a prolamin and a gelling agent. Furthermore, none of these references discloses or suggests using the combination of prolamin and gelling agent to achieve delayed-onset wherein the release of the active agent(s) is delayed for a period of time after the composition is administered.

WO 99/512,209 and WO 00/18447 to Ting et al. disclose a drug delivery system containing immediate-release compartment(s), which contains a compressed blend of an active agent and one or more polymers, enveloped by an extended-release compartment which contains a compressed blend of the active agent and hydrophilic and hydrophobic polymers. These publications disclose that the polymer of the drug system could be, among others, guar gum, gelatin, or zein. These references do not teach or suggest the present invention because (1) if Ting et al. use guar gum, gelatin, or zein in the core compartment, an immediate-release core compartment is achieved whereas the present invention relates to a sustained-release core; (2) alternatively, if Ting et al. use guar gum, gelatin, or zein in the envelop compartment, their extended-release coating composition is different from the delayed-onset coating composition of the present invention as the coating in the present invention is free of any active agents that are present in the core of the dosage unit.

There have been a limited number of references relating to using zein or other prolamins in a sustained-release core. As detailed below, none of these references teach or suggest the present invention.

British patent 935,672 to Sterling Drug Inc. discloses a sustained-release tablet composed of a solid medicament uniformly distributed in a matrix consisting essentially of an intimate mixture of zein and a solid hexitan tri-ester of a higher aliphatic acid, preferably sorbitan tristearate. Unlike the present invention, the gelling agent is absent in the matrix composition. The release profile obtained from such matrix composition has a big initial release in the gastric fluid followed by sharp drop to about 10% of initial rate.

U.S. Pat. No. 2,895,880 to Rosenthal is directed to a solid sustained-release pharmaceutical composition comprising a drug dispersed in a material selected from the group consisting of zein, gliadin, hordein and kafirin. The concentration of zein, gliadin, hordein or kafirin is about 20% to about 45% of the total weight of the formulation. This reference does not, however, teach or suggest using prolamins in combination with a gelling agent.

U.S. Pat. No. 3,258,768 to Klippel et al. discloses oral sustained-release pharmaceutical compositions comprising a tablet composed of a core comprising a medicament distributed in a matrix consisting of a compressed blend of a hydrophilic gum and corn protein and a shell comprising a medicament distributed in a matrix consisting of a compressed blend of hydrophilic gums. This reference discloses that the gum/corn protein weight ratio of the core compartment is between 1:1 to 1:2, which means the amount of gum in term of the combined weight of zein and gum is about 33% to about 50%. In contrast, as detailed in the sections to follow, the gum percentage in the mixture formed by zein and gelling agent is from about 0.5% to about 50%. Unlike the present invention, the composition disclosed in the reference achieves the roughly constant release rate in initial gastric fluid and in the intestinal fluid by creating a non-homogeneous distribution of the drug in the tablet. As illustrated by the examples in the reference, the drug concentration in the core is higher than in the shell thereby creating a non-homogenous distribution of the drug in the tablet.

U.S. Pat. No. 4,007,258 to Cohen et al. discusses a sustained-release pesticidal composition which includes a pesticide, a biological binding agent for the pesticide, and a matrix of a water-insoluble but water-swellable hydrophilic polymer. It discloses that the binder can be prolamins such as zein; glutelins such as gluten; scleroproteins such as collagen, gelatin, elastins and keratins; protamines; histones; and phosphoproteins such as casein and vitellin. However, the reference does not teach mixing a prolamin such as zein with a gelling agent such as gelatin. Moreover, it does not suggest the unexpected results achieved when zein and gelling agent are mixed.

U.S. Pat. No. 4,066,754 to Chou describes a composition for slowly releasing a veterinary medicament in a ruminant animal. Zein is a critical ingredient of this composition: it is utilized as a water-insoluble and water resistant binder when mixed with an organic solvent to granulated the powdered ingredient of the composition. Powdery zein is used at a critical level to slowly disintegrate the compressed bolus within the ruminant. This reference does not disclose using any gelling agent in combination with zein.

Katayama et al. [Drug Release From Dirctly Compressed Tablets Containing Zein, Drug Development and Industrial Pharmacy, 18(20), 2173-2184 (1992)] evaluated tablets prepared by the direct compression of spray-dried particles of a drug and zein and reported that the release of drug from the tablets was retarded compared with drug powder alone and tablets prepared from the physical mixtures. The drug release from the tablets was controlled by changing drug content and tablet weight. This reference does not teach or suggest the combination of prolamin and gelling agent or the unexpected results achieved by combining the two agents.

U.S. Pat. No. 4,308,251 to Dunn et al. describes a controlled-release tablet containing a release-controlling agent and an erosion-promoting agent. Suitable release-controlling agents include cellulose acetate phthalate, cellulose acetate derivatives disclosed in U.S. Pat. No. 2,196,768 to Hiatt, shellac, zein, acrylic resins, ethylcellulose, hydroxypropylmethylcellulose phthalate, sandarac, modified shellac, and so forth. Suitable erosion-promoting agents include corn starch, rice starch, potato starch and other equivalent vegetable starches, modified starch and starch derivatives, cellulose derivatives and modified cellulose or derivatives, e.g., methylcellulose, sodium carboxymethylcellulose, alginic acid and alginates, bentonite, veagum, cross-linked polyvinylpyrrolidone, ion-exchange resins, and gums, e.g., agar, guar, and so forth. However, this reference does not teach or suggest the present invention. As detailed in the sections to follow, in the present invention, as the amount of gelling agent such as xanthan gum and alginates increase in the composition, the erosion of the composition slows down. Therefore, in the present invention, gelling agents are not acting as erosion-promoting agents.

U.S. Pat. No. 4,424,235 to Sheth et al. discloses a hydrodynamically balanced controlled-release composition. Such composition contain, in percents by weight based on the total weight of the composition, from about 5% to about 80% of a hydrocolloid or mixture of hydrocolloids selected from the group consisting of acacia, gum tragacanth, locust bean gum, guar gum, karaya gum, agar, pectin, carrageen, soluble and insoluble alginates, methylcellulose, hydroxypropylmethylcellulose, hydroxypropylcellulose, sodium carboxymethylcellulose, carboxypolymethylene, gelatin, casein, zein and bentonite; up to about 60% of a fatty material or mixture of fatty materials, selected from the group consisting of beeswax, cetyl alcohol, stearyl alcohol, glyceryl monosteareate, hydrogenated caster oil and hydrogenated cottonseed oil and up to about 80% of edible inert pharmaceutical adjunct materials. Despite the disclosure of gelling agents (such as gelatin, locust bean gum) and zein, this reference does not specifically teach or suggest the gelling agent/prolamin ratio of the present invention and the unexpected results thereof.

U.S. Pat. No. 4,704,284 to Beatty et al. discloses a pharmaceutical tablet which releases an initial burst of therapeutic agent and thereafter releases the agent at an essentially constant rate. This reference discloses that excipients such as gelatin, natural gums, starches, modified starches, and alginates can be used. In addition, it also discloses that zein can be added as an excipient. However, this reference does not teach or suggest combining zein and gelling agent at the concentration range disclosed by the present invention. Furthermore, it does not teach or suggest the unexpected superior results achieved by the present invention.

U.S. Pat. No. 5,271,961 to Mathiowitz et al. discloses prolamin microspheres formed by phase separation in a non-solvent followed by solvent removal. There is no mention of combining a gelling agent with the prolamin.

European patent publication 158,277 (A2) is directed to an implantable composition containing zein as an excipient. It discloses that such implants can contain additional substance such as hydrophilic additives such as gelatin, sugar, polyalcohol and glycin. However, it does not disclose any specific concentration of gelatin when gelatin is optionally included. Nor does this reference teach or suggest that any unexpected results when gelatin is added.

U.S. Pat. No. 5,580,580 to Masterson et al. relates to a composition for once- or twice-daily use. It discloses a percutaneous sustained-release composition comprising gel forming agent such as zein and gelatin or mixtures thereof. However, the reference does not disclose the ratio of the zein and gelatin or the unexpected results achieved at the ratio disclosed by the present invention.

WO 96/14058 and U.S. Pat. No. 5,958,452 to Oshlack et al. disclose bioavailable sustained-release oral opioid analgesic dosage forms comprising a plurality of multiparticulates produced via melt extrusion techniques. These references disclose that zein could be one of the choices for hydrophobic materials used in the matrix and that such matrix can be encapsulated in a gelatin capsule. However, these references do not disclose any gelling agent being in the core matrix.

Several patents to Baichwal et al. disclose sustained-release formulations containing a gelling agent and optionally containing zein or other hydrophobic materials. For example, U.S. Pat. No. 5,399,359 and WO95/28916 disclose solid oral sustained-release drug formulations containing a gelling agent, an inert pharmaceutical diluent, a cationic cross-linking agent, and optionally zein. U.S. Pat. Nos. 5,455,046, 5,554,387, 5,667,801, and 5,846,563 disclose sustained-release formulation including a gelling agent, an inert pharmaceutical diluent, a medicament having moderate to poor solubility, an optional cationic cross-linking agent, and an optional hydrophobic material such as zein. U.S. Pat. No. 5,662,933 discloses a sustained release pharmaceutical formulation which includes a sustained-release excipient including a gelling agent, an inert pharmaceutical diluent, and an optional hydrophobic material such as zein. U.S. Pat. Nos. 5,773,025 and 6,048,548 disclose sustained-release oral solid dosage forms comprising agglomerated particles of a therapeutically active medicament in amorphous form, a gelling agent, an ionizable gel strength enhancing agent, an inert diluent, and an optional pharmaceutically acceptable hydrophobic material such as zein. U.S. Pat. No. 6,039,980 discloses a sustained-release formulation which includes from about 10 to about 40 percent or more by weight glactomamannan gum, from about 1 to about 20 percent by weight of an ionizable gel strength enhancing agent (e.g. organic salts such as sodium lactate, sodium citrate), an inert pharmaceutical filler and an optional hydrophobic material such as zein. Unlike the present invention, these patents to Baichwal et al. all teach or suggest that (1) zein is an optional component of the sustained-release formulation and not the key component of the sustained release matrix; (2) the role of zein is to slow down the hydration of the gelling agents; and (3) the amount of zein must be relatively low so that its presence does not disrupt the hydrophilic matrix formed upon exposure to an environmental fluid.

Citation or identification of any reference in this section shall not be construed as an admission that such reference is available as prior art to the present invention.

### SUMMARY OF THE INVENTION

The present invention relates to (1) a sustained-release composition comprising a prolamin, a gelling agent, and an active agent, and (2) a delayed-onset composition comprising a prolamin and a gelling agent.

More specifically, one embodiment of the present invention relates to a sustained-release composition comprising a prolamin, a gelling agent, and an active agent in the core of the sustained-release composition. Another embodiment of the present invention relates to a delayed-onset composition comprising a dry compressed coating comprising a prolamin and a gelling agent.

The sustained-release composition and the delayed-onset composition of the present invention can be used to deliver a variety of active agents (e.g., pharmaceutical agents), particularly pharmaceutical agents that are highly soluble in aqueous solutions, and/or are required to be delivered in high dosages.

The present invention may be understood more fully by reference to the following detailed description and illustrative examples which are intended to exemplify non-limiting embodiments of the invention.

### DESCRIPTION OF THE DRAWINGS

FIG. 1: Release profiles for 240 mg pseudo ephedrine HCl or sulphate tablets.

FIG. 2: Release profiles for 240 mg pseudoephedrine HCl or sulphate tablets.

FIG. 3: Release profiles for 100 mg tramadol® HCl tablets under standard dissolution conditions.

FIG. 4: Release profiles for 100 mg tramadol® hydrochloride tablets under various dissolution conditions.

FIG. 5: Release profiles for 100 mg tramadol® formulation, effect of surfactant in dissolution medium.

FIG. 6: Release profiles for various 100 mg tramadol® formulation s in 1% SDS dissolution medium.

FIG. 7: Release profiles for 24 mg betahistine 2HCl dry coated tablets.

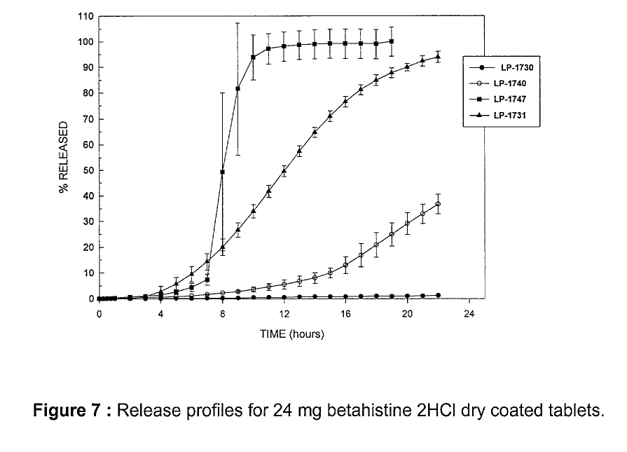

### DETAILED DESCRIPTION OF THE INVENTION

The present invention relates to a sustained-release composition comprising a prolamin, a gelling agent, and an active agent. The present invention also relates to a delayed-onset composition comprising a dry, compressed coating comprising a prolamin and a gelling agent.

In contrast to what is taught or suggested in any prior arts, the inventors of the present invention made a surprising discovery that when one or more prolamin(s) are mixed with from about 0.5% to about 50 wt. % of a gelling agent, the matrices formed are resistant to disintegration in a solution of detergent which is known to mimic intestinal fluid. It has further been found that such matrices are superior to others in their capacity at producing controlled release of very soluble drugs over long periods of time in a compact size. In addition, it has been found that a dry-compressed coating of prolamin(s) mixed with gelling agent(s) and inert ingredient(s) on an immediate-release core can be used to obtain a delayed-onset composition where a reproducible lag time for the release of the drug upon placement in an aqueous environment is followed by a rapid release of the drug. Such lag time can be several minutes, about 0.5 hours, about 1 hour, about 2 hours, about 4 hours, about 8 hours, about 12 hours, or about a day. These different lag time can be achieved by, for example, adjusting the ratio of the various components, and the thickness of the coating. Finally, it has also been found that such prolamin-based matrices can be used to obtain an initial release of a drug followed by a controlled release, which is desirable when the therapeutic effect must be achieved quickly after dosing. Such initial dose has been obtained in a reliable manner by coating the prolamin-based sustained-release composition with a film coating containing a certain percentage of the drug for immediate release.

It has also been found that when there is no gelling agent in the matrix, the composition disintegrates immediately in 1% sodium dodecyl sulfate(SDS) aqueous solution. As the concentration of the gelling agent increases, from 0 to 2.5%, to 5%, to 7.5%, to 9% of the total weight of the tablet, the erosion of the composition slows down and the rate of active agent release decreases as a result. This result is unexpected and counterintuitive. Prior to the present invention, one of ordinary skill in the art would have expected the opposite to happen, as gelling agents are usually hydrophilic and prolamins are hydrophobic. As the concentration of the hydrophilic component increases, one of ordinary skill in the art would have predicted an increase in the release rate of the active agent.

A. Prolamins

Prolamins are a group of simple proteins which yield only amino acid upon cleavage by enzymes or acids. They are grain-derived proteins that have very low solubility in water or neutral salt solvents. Prolamins are soluble in diluted acids and alkalies and in 70-90% alcohol. Prolamins are readily available and inexpensive, for example, as the by-product of grain processing. Prolamins are characterized by having a large number of hydrophobic amino acids, such as glutamine, asparagine and proline. Suitable prolamins include but are not limited to, zein (corn-derived prolamin), hordein (barley-derived prolamin), gliadin (wheat-derived prolamin), kafirin (sorghum-derived prolamin), secalinin (rye-derived prolamin), aveline (oat-derived prolamin), panincin (millet-derived prolamin), orzenin (rice-derived prolamin), and mixtures thereof.

The preferred prolamins for the present invention are zein, hordein, gliadin, and kafirin. The most preferred prolamin for the present invention is zein. The properties of zein are described in detail, for example, by L. C. Swallen in: “Zein—A New Industrial Protein”, Ind. and Eng. Chem., 33:394-398 (1941). In addition, zein is described in a monograph in the U.S. Pharmacopeia (USP) & National Formulary (NF) (USP 24 NF 19, page 2539).

Prolamins such as zein are commercially available. For example, zein can be purchased from Freeman Industries Inc. (Tuckahoe, N.Y., U.S.A.), and gliadin can be purchased from Sigma Chemicals Co. (St. Louis, Mo., U.S.A.).

B. Gelling Agents

Suitable gelling agents for the present invention include, but are not limited to, plant extracts, gums, synthetic or natural polysaccharides, polypeptides, alginates, synthetic polymers, or a mixture thereof.

Suitable plant extracts to be used as gelling agents include, but are not limited to, agar, ispaghula, psyllium, cydonia, ceratonia or a mixture thereof.

Suitable gums to be used as gelling agents include, but are not limited to, xanthan gum, guar gum, acacia gum, ghatti gum, karaya gum, tragacanth gum or a mixture thereof.

Suitable synthetics or natural hydrophilic polysaccharides to be used as gelling agents include, but are not limited to, hydroxyalkylcelluloses, cellulose ethers, cellulose esters, nitrocelluloses, dextrin, agar, carrageenan, pectin, furcellaran, starch or starch derivatives, cross-linked high amylose starch, or a mixture thereof.

Suitable polypeptides to be used as gelling agents include, but are not limited to, gelatin, collagen, polygeline or a mixture thereof.

Suitable alginates to be used as gelling agents include, but are not limited to, alginic acid, propylene glycol alginate, sodium alginate or a mixture thereof.

Suitable synthetic polymers to be used as gelling agents include, but are not limited to, carboxyvinyl polymer, polyvinyl alcohol, polyvinyl pyrrolidone, polyethelene oxide, polethylene glycols, copolymers of ethylene oxide and propylene oxide and their copolymers or a mixture thereof.

In a preferred embodiment, the gelling agent is a gum such as xanthan gum, guar gum, acacia gum, ghatti gum, karaya gum, tragacanth gum or a mixture thereof.

In a most preferred embodiment, the gelling agent is xanthan gum.

C. Active Agents

Suitable active agent for the present invention is any active agent that is desirable to be delivered in a sustained-release dosage form or in a delayed-onset form. Very often, such active agent is a pharmaceutical agent. However, active agent can also be non-pharmaceutical agents such as flavoring agents or fragrances. A comprehensive list of suitable pharmaceutical agents can be found in The Merck Index, 12th Edition. Preferably, the pharmaceutical agent is, but not limited to, pseudoephedrine hydrochloride, pseudoephedrine sulfate, acetaminophen or diclofenac sodium, verapamil, glipizide, nifedipine, felodipine, betahistine, albuterol, acrivastine, omeprazole, misoprostol, tramadol®, oxybutynin, trimebutine, ciprofloxacin, and salts thereof. In addition, the pharmaceutical agent can be an antifungal agent, such as ketoconazole, or an analgesic agent such as acetylsalicylic acid, acetaminophen, paracetamol, ibuprofen, ketoprofen, indomethacin, diflunisol, naproxen, ketorolac, diclofenac, tolmetin, sulindac, phenacetin, piroxicam, mefamanic acid, dextromethorphan, other non-steroidal anti-inflammatory drugs including salicylates, pharmaceutically acceptable salts thereof or mixtures thereof.

The solubility of the pharmaceutical agent in aqueous solution can be a wide variety of values. The aqueous solubility of the pharmaceutical agent can be less than 10⁻³ g/L, more than 10⁻³ g/L, more than 10⁻² g/L, more than 10⁻¹ g/L, more than 1 g/L, more than 10 g/L, more than 100 g/L, more than 500 g/L, more than 1000 g/L, or more than 2000 g/L. Preferably, the solubility is more than 100 g/L. More preferably, the solubility is more than 500 g/L. Most preferably, the solubility is more than 1000 g/L.

The pharmaceutical agent can meet a variety of dosage requirement. For example, the dosage requirement of the pharmaceutical agent can be less than 1 mg/dosage unit, more than 1 mg/dosage unit, more than 10 mg/dosage unit, more than 100 mg/dosage unit, more than 200 mg/dosage unit, more than 300 mg/dosage unit, more than 400 mg/dosage unit, more than 500 mg/dosage unit, or more than 1000 mg/dosage unit. Preferably, the pharmaceutical agent is more than 50 mg/dosage unit. More preferably, the pharmaceutical agent is more than 100 mg/dosage unit. Most preferably, the pharmaceutical agent is more than 200 mg/dosage unit.

D. Dissolution Profile of Sustained-Release Composition

The active agent of the composition exhibits the following in vitro dissolution profile when measured under standard dissolution condition according to USP 24, section 724, apparatus 1, 2, or 3:

(a) from about 0% to about 90% of said active agent is released between about 0 hour and about 4 hours of measurement;

(b) from about 10% to about 100% of said active agent is released between 0 hour and 8 hours of measurement;

(c) from about 20% to about 100% of said active agent is released between 0 hour and 12 hours of measurement; and

(d) from about 30% to about 100% of said active agent is released between 0 hour and 20 hours of measurement.

In a preferred embodiment, the active agent of the composition exhibits the following in vitro dissolution profile when measured under standard dissolution condition:

(a) from about 20% to about 90% of said active agent is released between about 0 hour and about 4 hours of measurement;

(b) from about 30% to about 100% of said active agent is released between 0 hour and 8 hours of measurement;

(c) from about 60% to about 100% of said active agent is released between 0 hour and 12 hours of measurement; and

(d) from about 70% to about 100% of said active agent is released between 0 hour and 20 hours of measurement.

In a more preferred embodiment, the active agent of the composition exhibits the following in vitro dissolution profile when measured under standard dissolution condition:

(a) from about 30% to about 80% of said active agent is released between about 0 hour and about 4 hours of measurement;

(b) from about 40% to about 90% of said active agent is released between 0 hour and 8 hours of measurement;

(c) from about 70% to about 100% of said active agent is released between 0 hour and 12 hours of measurement; and

(d) from about 80% to about 100% of said active agent is released between 0 hour and 20 hours of measurement.

In a most preferred embodiment, the active agent of the composition exhibits the following in vitro dissolution profile when measured under standard dissolution condition:

(a) from about 40% to about 70% of said active agent is released between about 0 hour and about 4 hours of measurement;

(b) from about 60% to about 90% of said active agent is released between 0 hour and 8 hours of measurement;

(c) from about 80% to about 100% of said active agent is released between 0 hour and 12 hours of measurement; and

(d) from about 90% to about 100% of said active agent is released between 0 hour and 20 hours of measurement.

E. Proportion

The prolamin is present at levels ranging from about 20 to about 90 wt. % of the total composition, preferably from about 25 to about 80 wt. % of the total composition, more preferably from about 30 to about 75 wt. % of the total composition, and most preferably from about 40 to about 70 wt. % of the total composition.

The gelling agent is present at levels ranging from about 1 to about 50 wt. % of the total composition, preferably from about 5 to about 40 wt. % of the total composition, more preferably from about 7.5 to about 30 wt. % of the total composition, and most preferably from about 9 to about 25 wt. % of the total composition.

The gelling agent is present at levels ranging from about 0.5 to about 50 wt. % of the combined weight of the gelling agent(s) and prolamin(s), preferably from about 1 to about 45 wt. % of the combined weight of the gelling agent(s) and prolamin(s), more preferably from about 2 to about 45 wt. % of the combined weight of the gelling agent(s) and prolamin(s), even more preferably from about 5 to about 45 wt. % of the combined weight of the gelling agent(s) and prolamin(s), and most preferably at about 10 to about 40 wt. % of the combined weight of the gelling agent(s) and prolamin(s).

F. Optional Components

The present prolamin-based sustained-release core compositions can optionally be coated with one or more coating layers. Any one of one or more coating layer(s) can be free of any pharmaceutical agent, or alternatively, the coating layer(s) can include one or more pharmaceutical agent(s). If a pharmaceutical agent is included in the one or more coating layers, such pharmaceutical agent may or may not be the same as the pharmaceutical agent in the core of the composition.

The present compositions may optionally further include a pharmaceutically acceptable carrier or vehicle. Such carriers or vehicles are known to those skilled in the art and are found, for example, in Remingtons's Pharmaceutical Sciences, 14th Ed. (1970). Examples of such carriers or vehicles include lactose, starch, dicalcium phosphate, calcium sulfate, kaolin, mannitol and powdered sugar. Additionally, when required, suitable binders, lubricants, and disintegrating agents can be included. If desired, dyes, as well as sweetening or flavoring agents can be included.

The present compositions may optionally include accessory ingredients including, but not limited to dispersing agents such as microcrystalline cellulose, starch, cross-linked poly(vinyl pyrrolidone), and sodium carboxymethyl cellulose; flavoring agents; coloring agents; binders; preservatives; surfactant and the like.

The sustained-release pharmaceutical composition, sustained-release exicipient composition, and the delayed-onset coating composition may further include a pharmaceutically acceptable carrier or vehicle. Such carriers or vehicles are known to those skilled in the art and are found, for example, in Remington's Pharmaceutical Sciences, 18th Ed. (1990). Examples of such carriers or vehicles include lactose, starch, dicalcium phosphate, calcium sulfate, kaolin, mannitol and powdered sugar. Additionally, when required, suitable binders, lubricants, disintegrating agents and coloring agents can be included. If desired, dyes, as well as sweetening or flavoring agents can be included.

Binders suitable for use in pharmaceutical compositions and exicipient include any binders known to one of ordinary skilled in the art.

Suitable forms of microcrystalline cellulose include, for example, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.). An exemplary suitable binder is a mixture of microcrystalline cellulose and sodium carboxymethyl cellulose sold as AVICEL RC-581. Suitable anhydrous or low moisture excipients or additives include AVICEL-PH-103™, Starch 1500 LM and CIPharm DC 93000.

Of course, the total amounts of all components would be 100 wt. %, and those of ordinary skill in the art can vary the amounts within the stated ranges to achieve useful compositions.

G. Routes of Administration

The composition of the present invention can be administered through, but not limited to, a number of routes such as oral, sublingual, rectal, and as an implant. The preferred route of administration of the compositions of the present invention is oral.

Compositions of the present invention that are suitable for oral administration may be presented as discrete units such as tablets or granules. Preferably, the compositions of the present invention are presented in a tablet form. Such tablets may be conventionally formed by compression or molding. Compressed tablets may be prepared by compressing in a suitable machine the mixture of one or more components described above. Molded tablets may be made by molding in a suitable machine the above components, which can be optionally moistened with an inert liquid diluent. The tablets may optionally be coated or scored, having indicia inscribed thereupon. A tablet can also be in a variety of forms, e.g., uncoated, dry coated, or film coated, etc. A tablet can also be in a variety of shapes (e.g., oval, sphere, etc.) and sizes. A comprehensive discussion of tablets can be found in references such as The Theory and Practice of Industrial Pharmacy by Lachman et al., 3rd Ed. (Lea & Febiger, 1986).

The present invention will be more readily understood by referring to the following examples which are given to illustrate the invention rather than limit its scope.

### EXAMPLES

Example 1 illustrates the present invention using sustained released Pseudoephedrine formulations. Example 2 illustrates the present invention using sustained released Tramadol® formulations. Example 3 illustrates dissolution profiles of the sustained-release composition in the sodium dodecyl sulfate (SDS) solution. Example 4 illustrates delayed-onset compositions comprises a prolamin-based coating.

The crosslinked high amylose starch used in the these examples are described in the pending U.S. patent application Ser. Nos. 09/028,385 and 09/257, 090. Lubritab® is a product made by Penwest Pharmaceuticals Co. (Cedar Rapids, Iowa, USA).

### EXAMPLE 1

### Preparation and Dissolution Profile of Controlled Released Pseudoephedrine Formulations

Pseudoephedrine formulations (Formulations LP-1584, LP-1712, LP-1422, LP-1598, and LP-1591) were prepared according to the recipes listed in Table 1. Formulations LP-1584, LP-1712, LP-1422, LP-1598, and LP-1591 are round and biconvex tablets with a diameter of 11.9 mm. Additionally, for comparison, Claritin-D® 24 hour tablet (manufactured by Schering Laboratories, USA) was used. Claritin-D® 24 hour tablet is a matrix formulation containing 240 mg of pseudoephedrine sulfate and a film coating with 10 mg loratadine. The overall mass of the tablet is 888 mg. The shape of the Claritin-D®24 hour tablet is oval. The length of the Claritin-D® 24 hour tablet is 18.3 mm, the width at center is 9.3 mm, and the thickness at center is 7.1 mm.

TABLE 1
Recipes for Controlled Released Pseudoephedrine Formulations

| Formulation | | | | | |
|---|---|---|---|---|---|
| Code | LP-1584 | LP-1712 | LP-1422 | LP-1598 | LP-1591 |
| Pseudoephedrine- | —HCl | -Sulphate | —HCl | —HCl | —HCl |
| Salt | | | | | |
| Amount of | 240 mg | 240 mg | 240 mg | 240 mg | 240 mg |
| Pseudoephedrine | | | | | |
| Salt in Equivalence | | | | | |
| to Pseudoephedrine | | | | | |
| Sulphate | | | | | |
| Cross-linked high | — | — | 232.8 mg | — | — |
| amylose starch | | | | | |
| Zein | 232.8 mg | 232.8 mg | — | — | 232.8 mg |
| Xanthan gum | 120 mg | 120 mg | — | — | — |
| hydroxypropyl- | — | — | 120 mg | 352.8 mg | 120 mg |
| methylcellulose | | | | | |
| (HPMC) K100M | | | | | |
| Lubritab ® | 6 mg | 6 mg | 6 mg | 6 mg | 6 mg |
| Silicon dioxide | 1.2 mg | 1.2 mg | 1.2 mg | 1.2 mg | 1.2 mg |
| portion of xanthan | 34% | 34% | — | — | 0 |
| gum in mixture of | | | | | |
| xanthan gum and | | | | | |
| zein | | | | | |
| Tablet Mass | about | 600 mg | about | about | about |
| | 600 mg | | 600 mg | 600 mg | 600 mg |

The Dissolution of Pseudoephedrine HCl or Pseudoephedrine Sulphate were measured under the following conditions:

Dissolution media:

USP standard buffer pH 1.2

USP standard buffer pH 6.8 (50 mM) and pH 7.5 (50 mM)

Dissolution conditions:

A USP type III apparatus was configured with three rows of dissolution vessels. The vessels were filled each with 250.0 g of dissolution medium. The cells containing the tablets were equipped with a 40 mesh screen in the lower caps and a 20 mesh screen in the upper caps. Dissolution tests were conducted at 37° C. using the method outlined below:

TABLE 2
Standard Dissolution Conditions

| | | Bacillus | |
|---|---|---|---|
| TIME | | Amylase | Agitation |
| (hours) | pH | (I.U./L) | (dips/min) |
| 00:30 | 1.2 | 0 | 15 |
| 00:30 | 6.8 | 4500 | 15 |
| 23:00 | 7.5 | 0 | 15 |

Each dissolution media was sampled at specific time points. Each aliquot was filtered through a 2 mm filter (Millex AP) prior to assay with a UV-Visible spectrophotometer (at wavelength 250 to 265 nm) or sampling for HPLC analysis.

Dissolution Profiles:

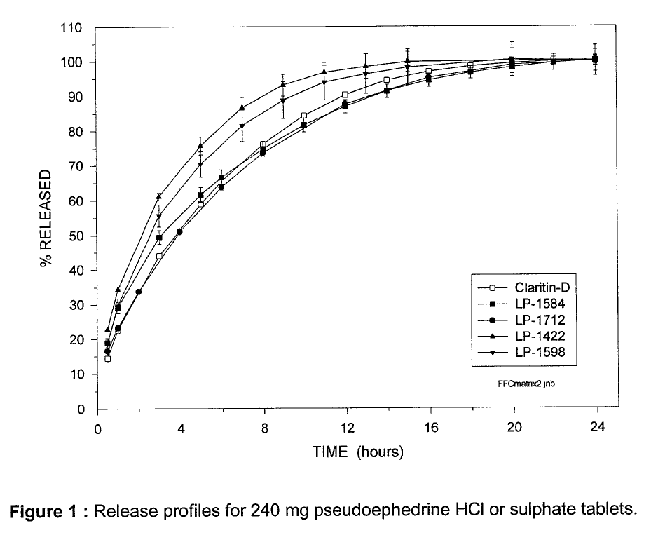

FIG. 1 compares the release profiles of various Pseudoephedrine matrix tablet compositions described in Table 1 under dissolution conditions described in Table 2. All tablets contain pseudoephedrine salts equivalent to 240 mg pseudoephedrine sulphate, and weigh about 600 mg. Claritin-D, LP-1584 and LP-1712 with zein and xanthan gum show very close dissolution profiles. LP-1712 containing pseudoephedrine sulphate is slower than LP-1584 containing pseudoephedrine HCl in the range 0 to 10 hrs. However, both profiles become superimposable after 10 hours. Formulations containing cross-linked high amylose starch and hydroxypropylmethylcellulose HPMC K100M or HPMC K100M alone release more rapidly than the reference tablets.

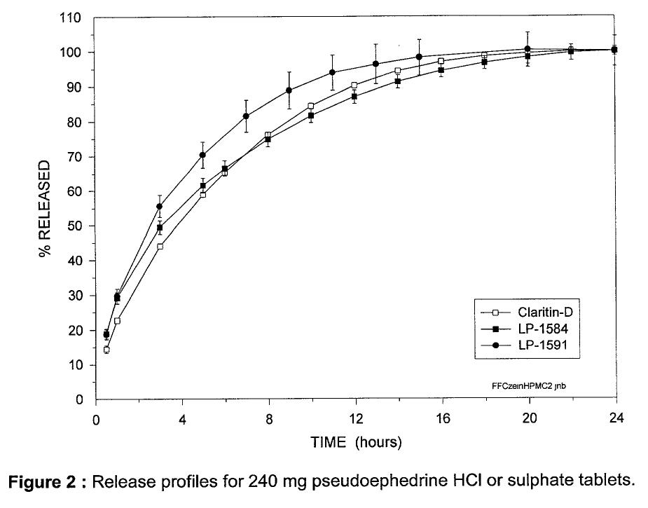

FIG. 2 compares the effect of using xanthan gum or HPMC K100 M as coexcipients with zein. The combination of zein with xanthan gum gives slower profiles.

### EXAMPLE 2

### Preparation and Dissolution Profile of Controlled Released Tramadol® HCl Formulations

TABLE 3
Description of the tramadol HCl formulations tested:

| | Formulation code | | | | | |
|---|---|---|---|---|---|---|
| | LP-1443 | LP-1695 | LP-1696 | LP-1582 | LP-1522 | LP-1707 |
| | mg | mg | mg | mg | mg | mg |
| Ingredients | (%, w/w) | (%, w/w) | (%, w/w) | (%, w/w) | (%, w/w) | (%, w/w) |
| Tramadol HCl | 100 | | | | | |
| | (30.77) | | | | | |
| Zein | — | — | — | 191.85 | 191.85 | 221.1 |
| | | | | (59.03) | (59.03) | (68.03) |
| Xanthan Gum | 29.25 | — | 221.10 | — | 29.25 | — |
| | (9.00) | | (68.03) | | (9.00) | |
| Excipient 3 | Cross- | HPMC | — | HPMC | — | — |
| | linked | K100M | | K100M | | |
| | high | 221.10 | | 29.25 | | |
| | amylose | (68.03) | | (9.00) | | |
| | starch | | | | | |
| | 191.85 | | | | | |
| | (59.03) | | | | | |

| | |
|---|---|
| Talc | 3.25 |
| | (1.00) |
| Silicon dioxide | 0.65 |
| | (0.20) |
| Tablet mass | 325 mg |
| Tablet | 9.53 mm |
| diameter | |

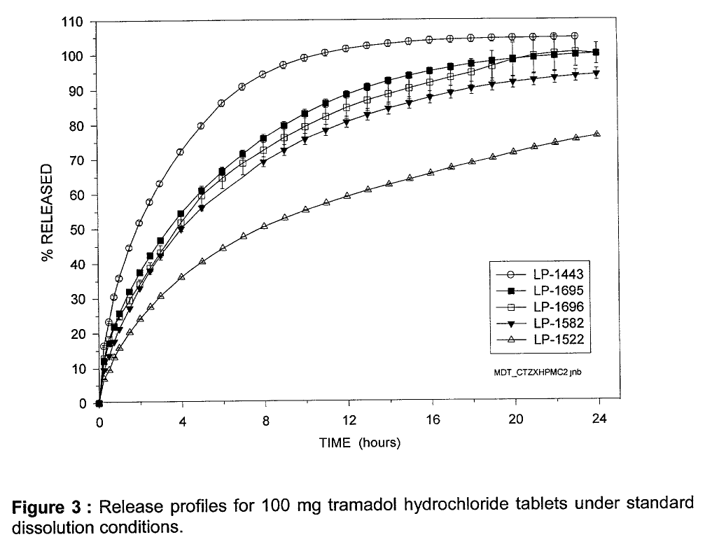

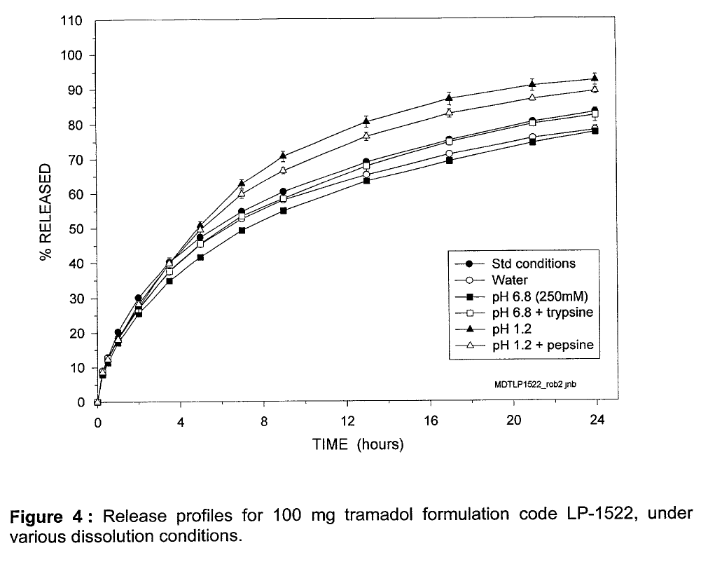

FIG. 3 shows the release profiles of the various tramadol HCl compositions described in Table 3 under the same dissolution conditions as in example 1 (Table 2). FIG. 4 shows the release profiles obtained for the zein-xanthan gum formulation (code LP-1522), obtained under various dissolution conditions. Dissolution at pH 6.8 with trypsine did not change the release profile when compared to standard dissolution conditions. A non-significant decrease in the release rate was observed in water, and at high ionic strength. Dissolution at pH 1.2 slightly accelerated the release. However, incorporation of pepsine at pH 1.2 did not further accelerate the release. The dissolution conditions are same as those described in Example 1.

For the formulation tested, neither trypsine nor pepsine had an effect on dissolution rate.

### EXAMPLE 3

### Effect of Sodium Dodecyl Sulfate (SDS) in Dissolution Media

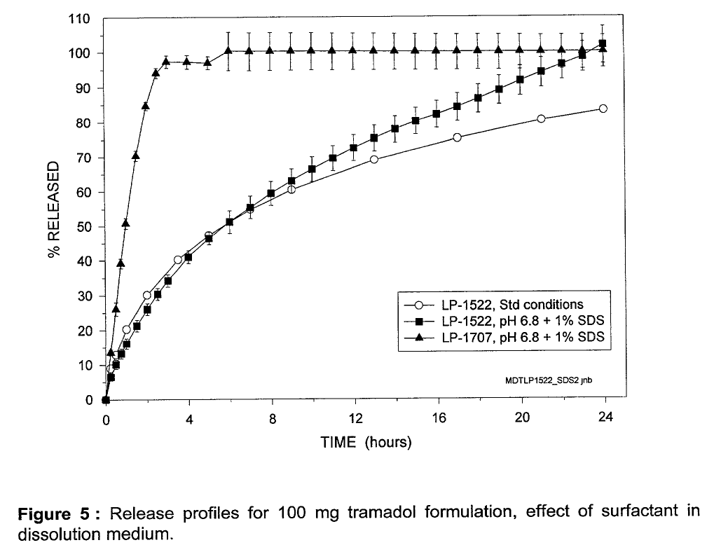

FIG. 5 shows the effect of incorporating 1% SDS in the dissolution medium for Formula LP-1522 and LP-1707 (compostion described above in Example 2). The formulation containing zein without a gel forming coexcipient, code LP- 1707, disintegrated rapidly in the presence of 1% SDS at pH 6.8 (simulating intestinal conditions). The same formulation did not disintegrate without surfactant, and slowly released about 70% tramadol® HCl in 24 hours (not shown). The zein formulation containing about 9% xanthan gum, code LP- 1522, did not disintegrate in the presence of 1% SDS. The release profile was generally similar to that obtained under standard dissolution conditions. Nevertheless, it can be concluded that a zein tablet does resist to simulated intestinal conditions upon incorporation of a gel forming coexcipient.

To further illustrate the effect of SDS on the dissolution profile of the various compositions, five different samples made according to Table 4 are made and their dissolution profiles compared.

TABLE 4
Tramadol ® Formulations:

| | Formulation code | | | | |
|---|---|---|---|---|---|
| | LP-1522 | LP-1955 | LP-1956 | LP-1957 | LP-1707 |
| | mg | mg | mg | mg | mg |
| Ingredients | (%, w/w) | (%, w/w) | (%, w/w) | (%, w/w) | (%, w/w) |
| Tramadol | 100 | | | | |
| HCl | (30.77) | | | | |
| Zein | 191.85 | 196.72 | 204.85 | 212.97 | 221.10 |
| | (59.03) | (60.53) | (63.03) | (65.53) | (68.03) |
| Xanthan | 29.25 | 24.375 | 16.25 | 8.13 | 0.0 |
| Gum | (9.00) | (7.5) | (5.0) | (2.5) | (0.0) |

| | |
|---|---|
| Talc | 3.25 |
| | (1.00) |
| Silicon | 0.65 |
| dioxide | (0.20) |
| Tablet mass | 325 mg |
| Tablet | 9.53 mm |
| diameter | |

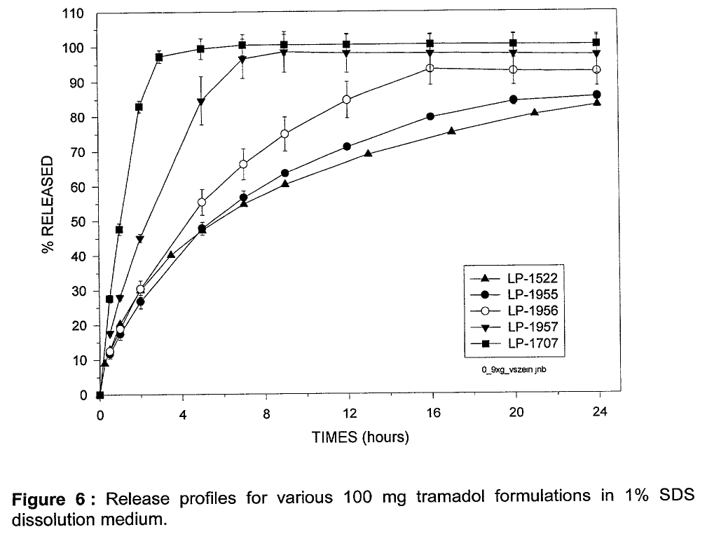

FIG. 6 shows the dissolution profiles of these five different formulations in dissolution media containing 1% SDS. FIG. 6 clearly demonstrates that as the concentration of the gelling agent (e.g., xanthan gum) increased from 0 to 9% of the total composition weight, the active agents release rates in 1% SDS were significantly reduced. Beyond 9% gelling agent, no further reduction of the release rate was observed.

### EXAMPLE 4

### Delayed-Onset Betahistine 2HCl Compositions

To illustrate that the combination of prolamin and gelling agent in a dry coating of a tablet can provide a delayed-onset dissolution profile, various 24 mg betahistine composition were prepared according to Table 5. All formulations tested were dry coated tablets, were all the drug was incorporated in the core, while the coating was used to control the lag time.

When the dry coating was composed of a (1:1) mixture of cross-linked high amylose starch and zein, less than 10% of the drug was released in the 0 to 7 hours time period. At 7 hours a burst occurred which allowed 100% of the drug to be dissolved at time 10 hours.

TABLE 5
Composition of the dry-coated betahistine 2HCL tablets.

| Formulation code | LP-1730 | LP-1740 | LP-1747 | LP-1731 |
|---|---|---|---|---|
| | mg | mg | mg | mg |
| Ingredients | (%, w/w) | (%, w/w) | (%, w/w) | (%, w/w) |
| Core | | | | |
| Betahistine 2HCl | 24.00 | 24.00 | 24.00 | 24.00 |
| | (24.00) | (24.00) | (20.00) | (24.00) |
| Cross-linked | 65.00 | 65.00 | 69.20 | 65.00 |
| high amylose | (65.00) | (65.00) | (57.67) | (65.00) |
| starch | | | | |
| Ac-Di-Sol | 10.00 | 10.00 | 25.60 | 10.00 |
| | (10.00) | (10.00) | (21.33) | (10.00) |
| Lubritab | 0.80 | 0.80 | 0.96 | 0.80 |
| | (0.80) | (0.80) | (0.80) | (0.80) |
| SiO2 | 0.20 | 0.20 | 0.24 | 0.20 |
| | (0.20) | (0.20) | (0.20) | (0.20) |
| Total | 100.00 | 100.00 | 120.00 | 100.00 |
| | (100.00) | (100.00) | (100.00) | (100.00) |
| Coating | | | | |
| Zein | 300.00 | 225.00 | 175.00 | 0 |
| | (100.00) | (75.00) | (50.00) | |
| Cross-linked | 0 | 75.00 | 175.00 | 395.00 |
| high amylose | | (25.00) | (50.00) | (79.00) |
| starch | | | | |
| HPMC K100M | 0 | 0 | 0 | 100.00 |
| | | | | (20.00) |
| Lubritab | 0 | 0 | 0 | 4.00 |
| | | | | (0.80) |
| SiO2 | 0 | 0 | 0 | 1.00 |
| | | | | (0.20) |
| Total | 300.00 | 300.00 | 350.00 | 500.00 |
| | (100.00) | (100.00) | (100.00) | (100.00) |
| Tabletmass | 400 mg | 400 mg | 470 mg | 600 mg |
| Tablet diameter | 10.32 mm | 10.32 mm | 10.32 mm | 10.32 mm |

The present invention is not limited in scope by the specific embodiments disclosed in these examples which are intended to illustrate the most preferred embodiments of the invention. Indeed, various modifications of the invention or other embodiments which are functionally equivalent to those shown and described herein will become apparent to those skilled in the art and are intended to be covered by the appended claims.

A number of references have been cited, the entire disclosures of which are incorporated herein by reference.

What is claimed:

1. A sustained-release composition comprising a core wherein said core comprises one or more active agents in combination with excipients consisting essentially of one or more prolamins and one or more gelling agents, and optionally containing one or more additional excipients.

2. The composition of claim 1, wherein said one or more gelling agents is about 0.5 wt. % to about 50 wt. % of the combined weight of said one or more prolamins and said one or more gelling agents.

3. The composition of claim 1, wherein said one or more prolamins is selected from the group consisting of zein, hordein, gliadin, kafirin, secalinin, aveline, panincin, orzenin, and mixtures thereof.

4. The composition of claim 1, wherein said one or more gelling agents is selected from the group consisting of plant extracts, gums, synthetic or natural polysaccharides, polypeptides, alginates, synthetic polymers, and mixtures thereof.

5. The composition of claim 1, wherein said one or more active agents is a pharmaceutical agent.

6. The composition of claim 5, wherein said pharmaceutical agent is selected from the group consisting of pseudoephedrine hydrochloride, pseudoephedrine sulfate, acetaminophen, diclofenac sodium, verapamil, glipizide, nifedipine, felodipine, betahistine, albuterol, acrivastine, omeprazole, misoprostol, tramadol, oxybutynin, trimebutine, ciprofloxacin, antifungal agents, analgesic agents, non-steroidal anti-inflammatory drugs including salicylates, pharmaceutically acceptable salts thereof and mixtures thereof.

7. The composition of claim 4, wherein said one or more gelling agent is xanthan gum.

8. The composition of claim 1, wherein said one or more additional excipients is selected from the group consisting of a starches, modified starches, and hydroxypropylmethylcellulose.

9. A delayed-onset composition comprises a core and a coating wherein said coating is dry-compressed and comprises expicients consisting essentially of one or more prolamins and one or more gelling agents, and optionally containing one or more additional exicpients.

10. The composition of claim 9, wherein said one or more prolamins is selected from the group consisting of zein, hordein, gliadin, kafirin, secalinin, aveline, panincin, orzenin, and mixtures thereof.

11. The composition of claim 9, wherein said gelling agent is selected from the group consisting of plant extracts, gums, synthetic or natural polysaccharides, polypeptides, alginates, synthetic polymers, and mixtures thereof.

12. The composition of claim 9, wherein said core comprises one or more active agents.

13. The composition of claim 12, wherein said one or more active agents is a pharmaceutical agent.

14. The composition of claim 13, wherein said pharmaceutical agent is selected from the group consisting of pseudoephedrine hydrochloride, pseudoephedrine sulfate, acetaminophen, diclofenac sodium, verapamil, glipizide, nifedipine, felodipine, betahistine, albuterol, acrivastine, omeprazole, misoprostol, tramadol, oxybutynin, trimebutine, ciprofloxacin, antifungal agents, analgesic agents, non-steroidal anti-inflammatory drugs including salicylates, pharmaceutically acceptable salts thereof and mixtures thereof.

15. The composition of claim 11, wherein said gelling agent is xanthan gum.

16. The composition of claim 10, further comprising an additional excipient.

17. The composition of claim 16, wherein said one or more additional excipients is selected from the group consisting of a starches, modified starches, and hydroxypropylmethylcellulose.
